(19) United States
(12) Patent Application Publication
(10) Pub. No.: US 2021/0177814 A1
(43) Pub. Date: Jun. 17, 2021

(54) USE OF GUANABENZ OR DERIVATES THEREOF FOR THE TREATMENT OF TYPE I IFN-DEPENDENT PATHOLOGIES

(71) Applicants: INSERM (Institut National de la Santé et de la Recherche Médicale), Paris (FR); Université d'Aix Marseille, Marseille (FR); Centre National de la Recherche Scientifque (CNRS), Paris (FR)

(72) Inventors: Jessica PEREGO, Marseille Cedex 09 (FR); Philippe PIERRE, Marseille Cedex 09 (FR); Evelina GATTI, Marseille Cedex 09 (FR)

(21) Appl. No.: 17/053,817

(22) PCT Filed: May 9, 2018
(86) PCT No.: PCT/IB2018/000609

§ 371 Date: Nov. 9, 2020

(51) Int. Cl. A61K 31/44; A61K 31/40; A61K 31/155; A61P 31/14

(52) U.S. Cl. CPC A61K 31/44; A61P 31/14; A61K 31/155; A61K 31/40

(57) ABSTRACT

The present invention relates to the use of Guanabenz or derivates thereof for the treatment of type I IFN-dependent pathologies. The inventors investigate here how pharmacological interference with the eIF2α-P pathway can be beneficial for the treatment of immune pathological conditions. Using both mouse and human DCs, as well as B cells, they show that GBZ prevents endosomal toll-like-receptor 9 (TLR9) activation by CpG ODN or DNA-Immunoglobulin complexes, as well as TLR3, TLR7 and RIG-I like receptors (RLR, RIG-I or MDA5), by RNAs or small compounds. In vivo, GBZ treatment protects mice from CpG-dependent cytokine shock and decreases anti-nucleic acid auto-antibodies production in the TMPD-induced systemic lupus erythematosus model. The present invention thus relates to a method of treating a type I IFN-dependent pathology in a subject comprising administering the subject with Guanabenz or a derivative thereof.

### FIELD OF THE INVENTION

The present invention relates to the use of Guanabenz or derivatives thereof for the treatment of type I IFN-dependent pathologies.

### BACKGROUND OF THE INVENTION

Dendritic cells (DCs) are regulators of the immune response whose immune-modulating functions, like antigen presentation, are considerably enhanced after detection of cell damage-associated or pathogen-associated molecular patterns (DAMPs & PAMPs). Toll-like receptors (TLRs) have a major role in PAMPs recognition and most TLRs detect their ligand at the cell surface, except TLR3, TLR7, TLR8 and TLR9 that are found in endosomes. TLR7 and TLR9, whose ligands are respectively ssRNA and DNA with unmethylated cytosine-phosphate-guanine (CpG) motifs, are expressed in a restricted number of immune cells, like B cell or plasmacytoïd DC (pDC), which specializes in the production of type-I interferon (IFN). In a pathological context, improper stimulation of endocytic TLRs by self-nucleic acids drives a feed-forward positive inflammatory amplification loop during which, B cell-dependent secretion of auto-antibodies and pDC activation by NA-Immunoglobulin (Ig) complexes fuels the continuous release of pathogenic levels of type-I IFN. TLR7- and TLR9-expressing pDCs and B cells are therefore key cellular players in the establishment and progression of several type-I IFN-dependent diseases such as systemic lupus erythematosus (SLE).

The signaling cascades that regulate ER homeostasis and promote cell survival are collectively known as the Integrated Stress Response (ISR). Recently, a cross-talk between TLRs signaling pathways and the ISR has been unravelled. Among the ISR signaling modules involved in this cross-talk, the phosphorylation of the α-subunit of eukaryotic translation initiation factor-2 (eIF2α) at Ser51 is particularly important (G. Clavarino, et al, Induction of GADD34 is necessary for dsRNA-dependent interferon-beta production and participates in the control of Chikungunya virus infection. PLoS Pathog 8, e1002708 (2012)). This phosphorylation inhibits the guanine nucleotide exchange factor eIF2B and results in reduced translation initiation and diminished production of newly synthesized proteins. Growth arrest and DNA damage inducible protein 34 (GADD34/PPP1R15A) is a key ISR inducible co-factor that reverses eIF2α phosphorylation through its association with phosphatase 1 catalytic subunit (PP1c) and restores normal protein synthesis after the translational arrest initiated by eIF2 kinases activation.

Guanabenz (GBZ) has been proposed to display specific GADD34 inhibitory activity by disrupting its interaction with PP1c (P. Tsaytler, et al., Selective inhibition of a regulatory subunit of protein phosphatase 1 restores proteostasis. Science 332, 91-94 (2011) and introduced as a model compound to protect cells from lethal protein misfolding and treat diseases like amyotrophic lateral sclerosis (I. Das et al, Preventing proteostasis diseases by selective inhibition of a phosphatase regulatory subunit. Science 348, 239-242 (2015). GBZ has also been reported to present some anti-inflammatory properties in different pathological situations, including multiple sclerosis. Very recently GBZ's capacity, and of its derivative Sephin 1, to inhibit specifically the GADD34-holophosphatase complex has been strongly challenged (A. Crespillo-Casado et al. PPP1R15A-mediated dephosphorylation of eIF2alpha is unaffected by Sephin1 or Guanabenz. Elife 6, (2017), and A Sephin1-insensitive tripartite holophosphatase dephosphorylates translation initiation factor 2α. J Biol Chem. (2018)) despite demonstration of increased eIF2α phosphorylation and clear proteostatic activity in treated cells.

### SUMMARY OF THE INVENTION

The present invention relates to the use of Guanabenz or derivatives thereof for the treatment of type I IFN-dependent pathologies. In particular, the present invention is defined by the claims.

### DETAILED DESCRIPTION OF THE INVENTION

The inducible phosphatase 1 co-factor GADD34/PPP1R15A that selectively mediates eIF2α-P dephosphorylation after endoplasmic reticulum stress has been shown to regulate pro-inflammatory cytokines and interferon expression in dendritic cells (DCs). Recently, several amino-guanidines compounds, such as Guanabenz (GBZ), have been shown to perturb the eIF2α phosphorylation-dephosphorylation cycle, and proposed to exert protective effects against protein misfolding and neurodegeneration. The inventors investigate here how pharmacological interference with the eIF2α-P pathway can be beneficial for the treatment of immune pathological conditions, such as type-I interferon-dependent auto-inflammatory diseases. Using both mouse and human DCs, as well as B cells, they show that GBZ prevents endosomal toll-like-receptor 9 (TLR9) activation by CpG ODN or DNA-Immunoglobulin complexes, as well as TLR3, TLR7 and RIG-I like receptors (RLR, RIG-I or MDA5), by RNAs or small compounds. In vivo, GBZ treatment protects mice from CpG-dependent cytokine shock and decreases anti-nucleic acid auto-antibodies production in the TMPD-induced systemic lupus erythematosus model.

Accordingly, the first object of the present invention relates to a method of treating a type I IFN-dependent pathology in a subject in need thereof comprising administering the subject with a therapeutically effective amount of Guanabenz or a derivative thereof.

As used herein, the term “subject” denotes a mammal, such as a rodent, a feline, a canine, and a primate. Preferably, a subject according to the invention is a human.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “type I interferon” or “type I IFN” has its general meaning in the art and refers to members of the type T interferon family of molecules. Examples of type I interferons are interferon alpha 1, 2a, 2b, 4, 5, 6, 7, 8, 10, 13, 14, 16, 17, 21, interferon beta, and interferon omega.

The term “type I IFN-mediated pathology” refers to any type I IFN inducible disease pathology i.e. a pathology caused by the overproduction of interferons and/or the overactivation of interferon-downstream genes. The type I interferon pathway has been implicated in the pathogenesis of a number of rheumatic diseases, including systemic lupus erythematosus (SLE), Sjögren syndrome, myositis, systemic sclerosis (SSc), Aicardi Goutières syndrome, Influenza A virus (IAV)-induced severe disease, juvenile idiopathic arthritis and rheumatoid arthritis. Moreover, increased expression of Type I interferons has been described in HIV infection, in transplant rejection, in graft versus host disease (GVHD), type 1 diabetes, inflammatory bowel diseases (ulcerative colitis and Crohn's disease), Graves' disease, microscopic polyangiitis, Wegener's granulomatosis, autoimmune thyroid diseases, glomerulonephritis and giant cell arteritis. Other examples of pathologies also include periodontal diseases (e.g. periodontitis). Diagnosis of said pathologies can be performed by any well-known method in the art. Even if type I IFNs in serum or plasma are not easily measured, type I IFN inducible genes can be conveniently measured improved sensitivity and specificity of diagnostic tests (Bengtsson et al., Lupus 9:664-671 (2000); Dall'era et al., Ann. Rheum. Dis. 64:1692-1697 (2005); Kirou et al., Arthritis Rheum. 50:3958-3967 (2004)). Several well defined type I IFN signatures have been used to correlate type I IFN activity with SLE or SSc disease pathogenesis (Eloranta et al., Ann. Rheum. Dis. 69:1396-1402 (2010)), and disease activity (Bilgic et al., Arthritis Rheum. 60:3436-3446 (2009)). The development of a type I IFN signature to identify subpopulations showing both activation and concordance of the type I IFN pathway between the peripheral blood and disease-affected tissues in both SLE and SSc (Higgs et al., Ann. Rheum. Dis. 70:2029-2036 (2011)) has demonstrated the potential utility of using a type I IFN signature as a marker in both diseases. In some embodiments, the type I IFN-mediated pathology is not a neurodegenerative disease characterized by inflammation. In some embodiments, the type I IFN-mediated pathology is not multiple sclerosis. In some embodiments, the type I IFN-dependent pathology is systemic lupus erythematosus.

In particular, Guanabenz and its derivatives are particularly suitable to prevent Toll-like receptor 3, 7 and 9 (TLR3, 7 and 9) activation by natural and synthetic agonists including RNA, CpG oligodeoxynucleotide or DNA-immunoglobulin complexes in endosomes. Moreover Guanabenz and its derivatives are particularly suitable for reducing titer of auto-antibodies in particular anti-nucleic acid autoantibodies. In particular, Guanabenz and its derivatives are particularly suitable to prevent formation of lipogranulomas. Moreover, Guanabenz and its derivatives are particularly suitable for decreasing the amount of type-I interferons, TNF-α and/or IL-6 cytokines. Guanabenz and its derivatives are particularly suitable for increasing the amount of IL-10 which will play an immuomodulatory role on inflammatory manifestations.

As used herein, the term Guanabenz (or GBZ) refers to 2-(2,6-dichlorobenzylidene)-hydrazinecarboximidamide, of formula (I):

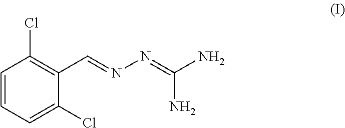 [SMILES: NC(N)=N/N=C/c1c(Cl)cccc1Cl]

As used herein, the term “Guanabenz derivative” refers to a compound that derives from the Guanabenz formula but preferably that does not exhibit activity toward the adrenergic a2A receptor relative to prior art compounds such as Guanabenz (i.e. the compound is not hypotensive).

In one embodiment, the Guanabenz derivatives used in the method of the present invention are mono-halogenated (hetero)aryl derivatives.

In one embodiments, the Guanabenz derivatives used in the method of the present invention are described in the international patent application WO2016/001390.

In one embodiments, the Guanabenz derivative is a compound of formula (I):

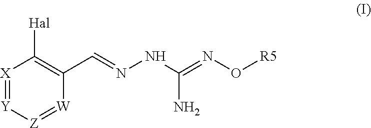

wherein:

Hal=F, CI, Br, I

X is either —CR1═ or —N═,

Y is either —CR2═ or —N═,

Z is either —CR3═ or —N═,

W is either —CR4═ or —N═,

R1 is selected from H, Hal, alkyl and O-alkyl;

R2 is selected from H, Hal, alkyl, O-alkyl and C(O)R6;

R3 is selected from H, Hal, alkyl and O-alkyl;

R4 is H, CI, F, I or Br;

R5 is H or alkyl, cycloalkyi, aralkyi, alkenyl, cycloalkenyl, heterocyclyl, aryl, C(O)-alkyl, and C(O)-aryl, each of which is optionally substituted with one or more R7 groups; R6 is selected from OH, O-alkyl, O-aryl, aralkyi, NH₂, NH-alkyl, N(alkyl)₂, NH-aryl, CF₃, alkyl and alkoxy;

each R7 is independently selected from halogen, OH, CN, COO-alkyl, aralkyi, heterocyclyl, S-alkyl, SO-alkyl, SO₂-alkyl, SO₂-aryl, COOH, CO-alkyl, CO-aryl, NH₂, NH-alkyl, N(alkyl)₂, CF₃, alkyl and alkoxy.

and wherein if Hal is CI and R4 is CI, then R5 is not H.

As used herein, the term “alkyl” includes both saturated straight chain and branched alkyl groups. Preferably, the alkyl group is a C₁₋₂₀ alkyl group, more preferably a C_{M S}, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl.

As used herein, the term “cycloalkyl” refers to a cyclic alkyl group. Preferably, the cycloalkyl group is a C₃₋₁₂ cycloalkyl group.

As used herein, the term “alkenyl” refers to a group containing one or more carbon-carbon double bonds, which may be branched or unbranched. Preferably the alkenyl group is a C₂₋₂₀ alkenyl group, more preferably a C₂-i₅ alkenyl group, more preferably still a C₂-i₂ alkenyl group, or preferably a C₂₋₆ alkenyl group, more preferably a C₂₋₃ alkenyl group. The term “cyclic alkenyl” is to be construed accordingly.

As used herein, the term “aryl” refers to a C₆-i₂ aromatic group. Typical examples include phenyl and naphthyl etc.

As used herein, the term “heterocycle” (also referred to herein as “heterocyclyl” and “heterocyclic”) refers to a 4 to 12, preferably 4 to 6 membered saturated, unsaturated or partially unsaturated cyclic group containing one or more heteroatoms selected from N, O and S, and which optionally further contains one or more CO groups. The term “heterocycle” encompasses both heteroaryl groups and heterocycloalkyl groups as defined below.

As used herein, the term “heteroaryl” refers to a 4 to 12 membered aromatic which comprises one or more heteroatoms. Preferably, the heteroaryl group is a 4 to 6

membered aromatic group comprising one or more heteroatoms selected from N, O and S. Suitable heteroaryl groups include pyrrole, pyrazole, pyrimidine, pyrazine, pyridine, quinoline, thiophene, 1,2,3-triazole, 1,2,4-triazole, thiazole, oxazole, iso-thiazole, iso-oxazole, imidazole, furan and the like.

As used herein, the term “heterocycloalkyl” refers to a 3 to 12 membered, preferably 4 to 6 membered cyclic aliphatic group which contains one or more heteroatoms selected from N, O and S. N-containing 5 to 6 membered heterocycloalkyl are preferred. Preferred heterocycloalkyl groups include piperidinyl, pyrrolidinyl, piperazinyl, thiomorpholinyl and morpholinyl. More preferably, the heterocycloalkyl group is selected from N-piperidinyl, N-pyrrolidinyl, N-piperazinyl, N-thiomorpholinyl and N-morpholinyl.

As used herein, the term “aralkyl” includes, but is not limited to, a group having both aryl and alkyl functionalities. By way of example, the term includes groups in which one of the hydrogen atoms of the alkyl group is replaced by an aryl group, e.g. a phenyl group. Typical aralkyl groups include benzyl, phenethyl and the like.

In one embodiment, the Guanabenz derivative is a compound of formula (II):

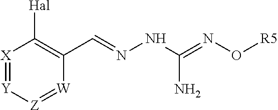

wherein:

Hal=F, CI, Br, I

X is either —CR1═ or —N═,

Y is either —CR2═ or —N═,

Z is either —CR3═ or —N═,

W is either —CR4═ or —N═,

R1 is selected from H, Hal, alkyl and O-alkyl;

R2 is selected from H, Hal, alkyl, O-alkyl and C(O)R6;

R3 is selected from H, Hal, alkyl and O-alkyl;

R4 is H, CI, F, I or Br;

R5 is H or alkyl, cycloalkyi, aralkyi, alkenyl, cycloalkenyl, heterocyclyl, aryl, C(O)-alkyl, and C(O)-aryl, each of which is optionally substituted with one or more R7 groups;

R6 is selected from OH, O-alkyl, O-aryl, aralkyi, NH₂, NH-alkyl, N(alkyl)₂, NH-aryl, CF₃, alkyl and alkoxy;

each R7 is independently selected from halogen, OH, CN, COO-alkyl, aralkyi, heterocyclyl, Salkyl, SO-alkyl, SO₂-alkyl, SO₂-aryl, COOH, CO-alkyl, CO-aryl, NH₂, NH-alkyl, N(alkyl)₂, CF₃, alkyl and alkoxy;

In one embodiment, the guanabenz derivative is selected from group consisting of:

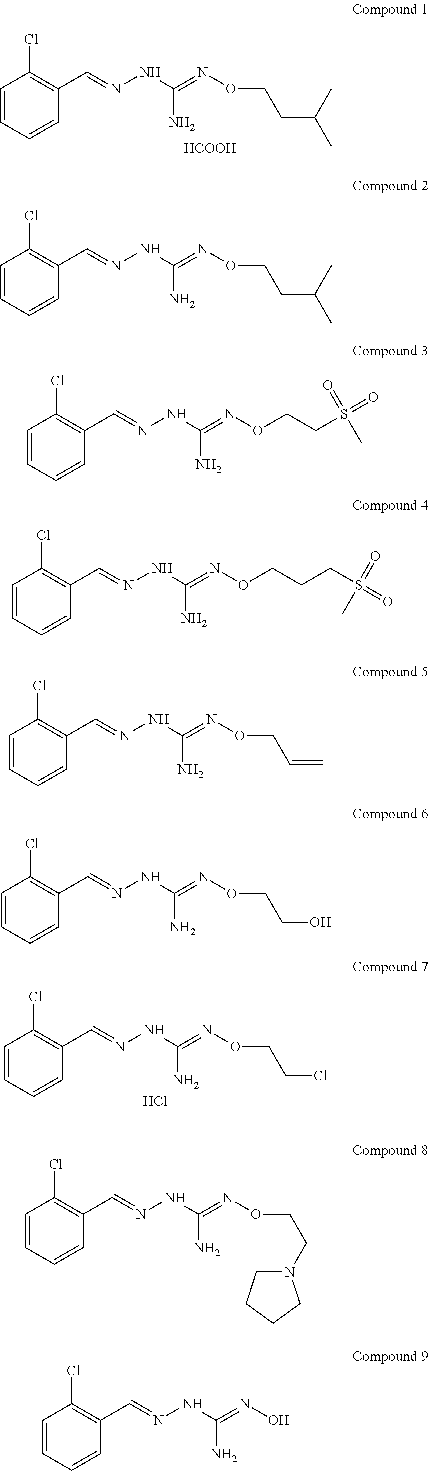

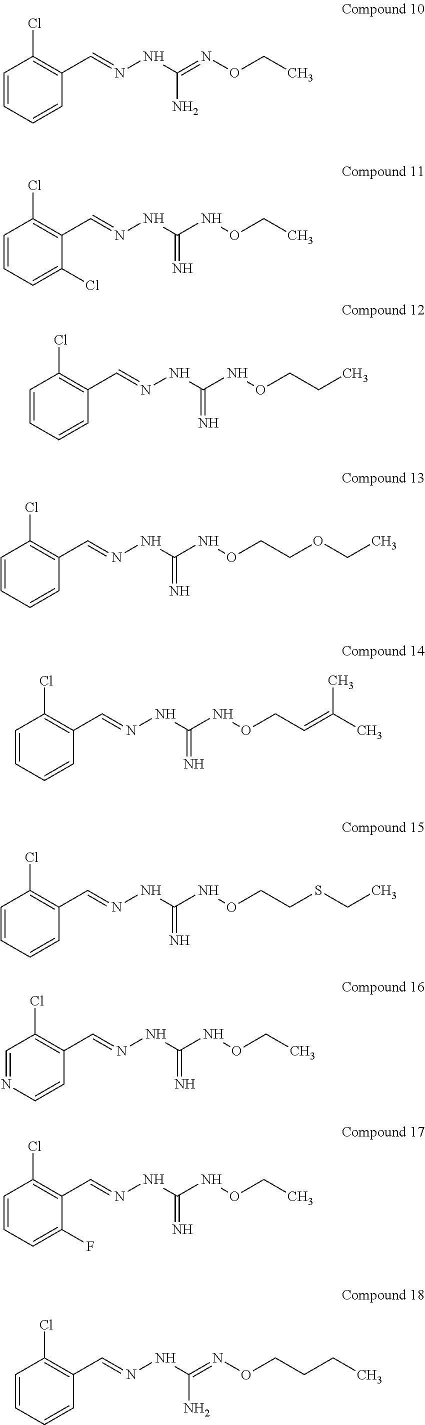

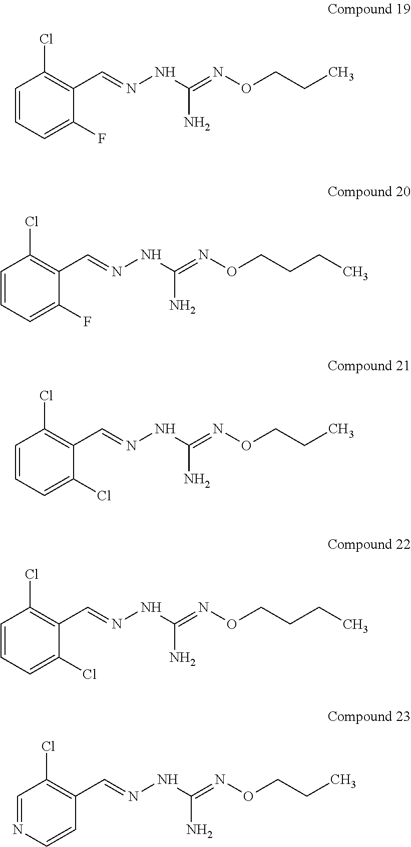 [SMILES: CCCO/N=C(\N)N/N=C/c1c(F)cccc1Cl ; CCCCO/N=C(\N)N/N=C/c1c(F)cccc1Cl ; CCCO/N=C(\N)N/N=C/c1c(Cl)cccc1Cl ; CCCCO/N=C(\N)N/N=C/c1c(Cl)cccc1Cl ; CCCO/N=C(\N)N/N=C/c1ccncc1Cl]

and pharmaceutically acceptable salts thereof.

In one embodiment, the guanabenz derivatives used in the method of the present invention is described in the following patent applications: U.S. Pat. No. 3,982,020; US 2004/0068017; WO 2008/061647; WO2005/031000; EP1908464; U.S. Pat. No. 7,932,422; EP2076253; WO/2008/041134; US20170247344; US20170151196; US20170152220; WO2007060342; WO2008041133; US20090306430.

In some embodiments, guanabenz or derivatives thereof is administered to the subject with a therapeutically effective amount.

The terms “administer” or “administration” refer to the act of injecting or otherwise physically delivering a substance as it exists outside the body (e.g., guanabenz or derivatives thereof) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of guanabenz or derivatives thereof for use in a method for the treatment of autoimmune diseases at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The compositions according to the invention are formulated for parenteral, transdermal, oral, rectal, intrapulmonary, subcutaneous, sublingual, topical or intranasal administration. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. In a one embodiment, the compositions according to the invention are formulated for oral administration. In a one embodiment, the compositions according to the invention are formulated for intravenous administration. In a one embodiment, the compositions according to the invention are formulated for topical administration. Typically the active ingredient of the present invention (i.e. guanabenz or derivatives thereof) is combined with pharmaceutically acceptable excipients, and optionally sustained-release matrices, such as biodegradable polymers, to form pharmaceutical compositions. The term “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports.

In some embodiments, Guanabenz or derivatives thereof is administered to the subject in combination with another active ingredient. In some embodiments, Guanabenz or derivatives thereof of the present invention is administered to the subject in combination with a standard treatment of the considered autoimmune disease.

The present invention further relates to a pharmaceutical composition comprising Guanabenz or derivatives thereof.

The invention will be further illustrated by the following figures and examples. However, these examples and figures should not be interpreted in any way as limiting the scope of the present invention.

### FIGURES

FIG. 1: Guanabenz inhibits TLR3 and TLR9 activation in mouse Flt3-L DCs and human blood pDCs. A) Sorted bone marrow derived Flt3L-DCs were activated for 6 h with 10 μg/mL poly I:C LMW (TLR3 in DC1) or 1 μM ODN 1585 (TLR9 in pDCs). GBZ was added at 50 μM and inhibited IFN-β and Interleukin-6 (IL-6) production in activated cells. B) hTLR4, hTLR3, hTLR9 and hTLR7 expressed in the reporter HEK-Blue™ cells are inhibited by GBZ. TLR-expressing HEK-Blue™ cells were respectively activated with 100 ng/mL LPS, 10 μg/mL poly I:C LMW, 2.5 μM ODN 2006 or 5 μg/mL imiquimod, respectively. GBZ was added at 50 μM. TLR activity was measured indirectly through SEAP activity as absorbance at 405 nm. GBZ inhibited human TLR3, TLR7 and TLR9 signaling, but not TLR4._C) Human primary pDCs were isolated from blood and activated either with 3 μM CpG ODN 2216 (top) or with 2% of anti-DNA IgG containing serum from lupus patients (bottom). GBZ was added at 50 μM. Protein concentration of IFN-α and TNF-α secreted were measured at the indicated time points or at 16 h. Statistical significance was assigned using two tailed t-test on at least n=3 independent experiments (* p<0.05; ** p<0.005; ***p<0.0005).

FIG. 2: Interferon β (IFNb) production in response to cytosolic dsRNA (Poly I:C) in WT and Gadd34-deficient embryonic fibroblasts (MEFs) treated with Guanabenz.

(A) IFNb expression in response to lipofected dsRNA (poly I:C) was measured by qPCR (mRNA) or (B) by ELISA (secreted protein) after treatment with Guanabenz (GBZ). (C) Activation of the transcription IRF3 by phosphorylation that drives IFN-β mRNA expression was monitored by western blot in WT and Gadd34-deficient animals (ΔC/ΔC, KO or −/−) in response to lipofected poly I:C in presence of GBZ. (D) eIF2α A/A mutants MEFs (unable to phosphorylate eIF2α on Serine 52, that is targeted by GADD34 and to inhibit protein synthesis) were also tested for IFN-β production and IRF3 activation. This results indicates that Guanabenz is able to inhibit the RIG-I like (RLR) dsRNA sensing pathway and to inhibit IRF3 activation as well as Interferon-β expression both at the transcriptional and translational level, this independently of GADD34 activity.

FIG. 3: Guanabenz rescues mice from TLR9-dependent cytokines shock.

WT and GADD34^{ΔC/ΔC} mice were injected with D-galN (20 mg/mouse), CpG ODN 1826 or PBS for control mice (50 μg/20 g), GBZ or clonidine (2 mg/kg). (A) Survival of C57/BL6 mice is represented as total percentage of survival after 48 h or in a Kaplan-Meier plot. (B) Survival of FVB (WT) and FVB GADD34^{ΔC/ΔC} mice is represented as total percentage of survival after 48 h or in a Kaplan-Meier plot. (C) The concentration of circulating TNF-α, IL-10 and IL-6 in C57/BL6 mice was measured after 1 h and/or 3 h of CpG ODN 1826. (D) Liver tissue damage and histology score were blindly determined by a professional anatomopathologist. Statistical significance was assigned on sample comparison by a one-way ANOVA, followed by Tukey range test and on the survival curve using log-rank test, followed by Benjamini-Yekutieli correction (* p<0.05; ** p<0.005; ***p<0.0005.).

FIG. 4: Guanabenz lowers lupus-like symptoms in the pristane-injection model.

At day 7 mice were injected with CpG ODN 1585 (50 μg/20 g). Blood was collected once a month to dose the circulating autoantibodies level. Mice were sacrificed at week 24 after disease induction. (A) Flow cytometry analysis of peritoneum exudate cells populations (PEC) at day 14. (B) Mice were sacrificed at week 24 and gene expression of ISGs and cytokines (TNF-α) was determined by qPCR on PEC cells. Statistical significance was assigned using two tailed Student's t test (* p<0.05). (C) Level of circulating anti-RNA antibodies (normalized to control mice), anti-nuclear antibodies (arbitrary units), presence of lipogranulomas in the peritoneal cavity (arbitrary score) and glomerulopathy (histological score blindly determined by an anatomopathologist) at week 24. Statistical significance was assigned by a one-way ANOVA, followed by Tukey range test (* p<0.05;** p<0.005; ***p<0.0005.). N.S., not significant. n values in the figure indicate the number of mice.

### EXAMPLE

### Material & Methods

### Cells Culture

BM-derived DCs were differentiated in vitro from the bone marrow of 6-8 weeks old male mice, using either FLT3 ligand or GM-CSF. Flt3L-DCs were used for experiments between days 6 and 7. Flt3L was produced using B16-Flt3L hybridoma cells. Bone marrow progenitors were plated at 1.5 10⁶ cells/ml, 4 ml/well in 6 well plate, and cultivated with RPMI (GIBCO), 10% FCS (Sigma Aldrich), 100 units/ml penicillin and 100 μg/ml streptomycin (GIBCO), 50 μM b-mercaptoethanol (VWR) and Flt3L. For sorted Flt3L-DCs, cells were gently harvested at day 7 with cold PBS 2% FCS, centrifuged 1200 rpm 5 min at 4° C., counted and stained with for 30 mins at 4° C. Cells were sorted with the FACS ARIASorp. Cells were kept on ice all the time. After sorting, cells were resuspended in supplemented Flt3L-DCs medium at the concentration of 0.2 0.10⁶ cells/well and plated at in a 96 wells plate U bottom. GM-CSF DCs were produced and used for experiments at day 6 of differentiation.

### Reagents and Molecular Biology

ODN 2006, 2216, 1585 and 1826, Poly I:C High and Low Molecular Weight are from InvivoGen, San Diego, Calif.; lipopolysaccharide (Escherichia coli 055:B5), clonidine, chloroquine, D-(+)-Galactosamine hydrochloride and 25-HC are from Sigma Aldrich; Guanabenz is from Tocris Bioscience. For quantitative PCR, total RNAs were extracted and purified using the RNeasy Mini Kit (Qiagen). 100 ng to 1 μg of total RNA were subjected to reverse transcription using SuperScript II. Each gene transcripts were quantified by SYBR Green method with 7500Fast (Applied Biosystems). The relative amount of each transcript was determined by normalizing to internal housekeeping gene expression.

### Mouse Splenic Cells

Splenocytes were dissociated injecting Liberase TL, followed by 25 min incubation at 37° C. Cells were washed using MACS buffer (PBS 1X+1% FBS+2 mM EDTA) and passed in a cell strainer 70 μm, then centrifuged 5 min at 450 RCF, 4° C. Red blood cells were lysed using the commercial buffer from eBioscience. Dendritic cells were purified using CD11c⁺ positive selection kit from Miltenyi. Splenic DCs were then resuspended in the same medium as for Flt3L-DCs at 1.10⁶ cells/ml and plated in a 12 wells plate, 2 ml×well. B lymphocytes were purified using B cell isolation kit, a negative selection kit from Miltenyi. B cells were then resuspended in a medium composed of RPMI (GIBCO), 10% FCS (Sigma Aldrich), lx glutamine, 1× non-essential amino acids, 10 mM HEPES (all the previous from GIBCO), 50 μM b-mercaptoethanol (VWR). Cells were plated at 1×10\hat{ }6 cells/well in 96 well plate, flat.

Human pDCs and B Cells

Human PBMCs were isolated from whole blood by density gradient using Ficoll-Paque PLUS (GE Healthcare), followed by a density gradient of Percoll PLUS (GE Healthcare), to separate the lymphocytic fraction, containing B cells, from the monocytic lineage fraction, containing pDCs. Both cell types were isolated using a negative selection kit from Miltenyi: B cell isolation II human (for B cells) and plasmacytoid dendritic cells isolation II human (for pDCs). pDCs were cultured at 0.5 10\hat{ }6 cells/mL in RPMI 1640 medium containing 10% FCS and complemented with IL-3 at 10 ng/mL. B lymphocytes were cultured at 0.5 10\hat{ }6 cells/mL in RPMI 1640 medium containing 10% FCS and complemented with 100 units/ml penicillin and 100 μg/ml streptomycin (GIBCO) and 1×L-glutamine (GIBCO). Both cell types were plated in a 96 well plate, U bottom.

### Cell Lines

HEK 293 hTLRs were grown in DMEM, with the addition of 10% FCS (Sigma Aldrich) and the proper selection antibiotics. A20 cells were grown in RPMI (GIBCO), 10% FCS (Sigma Aldrich), 50 μM b-mercaptoethanol (VWR). Cal-1 were grown in RPMI (GIBCO), 10% FCS (Sigma Aldrich), 2 mM L-glutamine, 1× non-essential amino acids, 10 mM HEPES, 1 mM sodium pyruvate (all the previous from GIBCO). For performing experiments, cells were plated 16 h before in a 12 wells plate at 1×10\hat{ }6 cells/mL, 1 ml×well, in a complete medium with 1% FCS. All cell lines were mycoplasma free and kept at 37° C. and 5% CO2.

### Flow Cytometry

Cell suspensions were incubated with the antibodies cocktail diluted in cold FACS buffer (PBS, 2% FCS, 2 mM EDTA) for 30 minutes at 4° C. For intracellular staining, cells were permeabilised with Fix & Perm kit (BD Biosciences). Flow cytometry was conducted using LSR 561 machine (BD Biosciences) and data were analyzed with FlowJo (Tree Star).

### Confocal Microscopy and PLA Assay

Cal-1 cells were harvested, centrifuged 5 min at 4° C. 1200 RPM, then resuspended in pre-warmed medium serum-free and drop on a 12 mm coverslips, covered with 1% alcyan blue. The coverslips where then incubated for 20 mins at 37° C. and fixed with 3% paraformaldehyde. Proximity Ligation Assay was then performed using the Sigma Aldrich Duolink kit, according to the manufacturer's instruction. The primary antibodies used are: anti-TLR9 (H-100) Santa Cruz, rabbit polyclonal (1:50); anti-Vamp3 (N-12), Santa Cruz, goat polyclonal (1:100); anti-human MyD88, R&D system, goat polyclonal (1:100); anti-human IRF7 (G-8), Santa Cruz, mouse IgG2a (1:100). Duolink PLA probe rabbit PLUS and goat or mouse MINUS, conjugated with oligonucleotides, were used as secondary antibodies. The samples were incubated in the ligation solution consisting of Duolink Ligation stock (1:5) and Duolink Ligase (1:40). Detection of the amplified probe was done with the Duolink Detection Kit Orange. The images were taken with the confocal microscope LSM580 (Carl Zeiss), 63× objective and accompanying imaging software.

### Gene Expression Analysis

Total RNA was isolated with RNeasy kit (Qiagen). cDNA was synthesized with random hexamers and superscript II reverse transcriptase (Invitrogen). Quantitative real-time PCR analysis was performed with Applied Biosystems PRISM 7700 Sequence Detection System. For Affymetrix microarray analysis, GM-CSF DCs were cultured in RPMI supplemented with 5% FCS, 50 μM beta-mercaptoethanol and GM-CSF. Cells differentiated for 6 days were treated for 8 h with microbial stimuli and harvested before lysis. Control and Proteus mirabilis treated DCs were incubated with the bacteriostatic chloramphenicol to avoid bacterial growth. Guanabenz was used at 50 μM. Hybridization to arrays (Affymetrix GeneChip Mouse Gene 1.0ST) and image scanning were performed according to the Affymetrix Expression Analysis Technical Manual. Gene Expression microarray raw data were normalized using limmaGUI software (R/Bioconductor, Boston, Mass., USA). Data can be accessed through the GEO repository accession number GSE90831.

### Translation Intensity Measurement

Puromycin labelling for measuring the intensity of translation was performed. Puromycin (Sigma, min 98% TLC, cell culture tested, P8833, diluted in PBS) was added at 2.5 g/ml in the culture medium and the cells were incubated for 15 min at 37° C. and 5% CO2. Cells were fixed and permeabilized with cytofix/cytoperm buffer (BD Biosciences) and stained with the anti-puromycin 12D10 antibody directly coupled with Alexa 488, diluted in Perm/Wash buffer (BD Biosciences). Flow cytometry was conducted using LSR 561 machine (BD Biosciences) and data were analyzed with FlowJo (Tree Star).

### Immunoblotting

Cells were lysed in 1% Triton X-100, 50 mM Hepes, 10 mM NaCl, 2.5 mM MgCl2, 2 mM EDTA, 10% glycerol, supplemented with Complete Mini Protease Inhibitor Cocktail Tablets (Roche). Protein quantification was performed using the BCA Protein Assay (Pierce). 20-25 μg of Triton X-100-soluble material were loaded on 10% SDS-PAGE before immunoblotting and chemiluminescence detection (SuperSignal West Pico Chemiluminescent Substrate, Pierce). Rabbit polyclonal antibodies against eIF2α (D7D3), was purchased from Cell Signalling Technologies. Rabbit polyclonal antibody against P-eIF2α (Ser 51) was from Abcam. Mouse monoclonal antibodies against β-actin was from Sigma and Upstate respectively. Secondary antibodies were purchased from Jackson ImmunoResearch Laboratories.

### Cytokines Measurement

Mouse IFN-β quantification in culture supernatant was performed using the Mouse Interferon Beta ELISA kit (PBL InterferonSource); mouse IL-6, TNFα, IL-10, human TNFα and human IFNα were quantified using ELISA kit (eBioscience) respectively, according to manufacturer instructions.

### Animal Studies

Wild type C57BL/6 mice were purchased from Jackson Laboratories. GADD34^{ΔC/ΔC} mice (FVB background) and wild type littermate were originally obtained from L. Wrabetz (San Raffaele Institute, Milan) and maintained in the animal facility of CIML under specific pathogen-free conditions. This study was carried out in strict accordance with the recommendations in the Guide for the Care and Use of Laboratory Animals the French Ministry of Agriculture and of the European Union. Animals were housed in the CIML animal facilities accredited by the French Ministry of Agriculture to perform experiments on alive mice. All animal experiments were approved by Direction Départementale des Services Vétérinaires des Bouches du Rhône (Approval number A13-543). All efforts were made to minimize animal suffering.

### TMPD Model

Wild type female mice C57BL/6 of 6 weeks of age were injected i.p. with 500 μL of TMPD (Sigma Aldrich) or PBS. From day 1 to day 13 mice were injected i.p. every 2 days with GBZ or PBS/DMSO 2 mg/kg. Mice were injected i.p. at day 7 with ODN 1585 or PBS 50 μg/20 g of mouse. Mice were sacrificed at day 14 or after 6 months. At day 14, peritoneal exudate cells (PEC) were taken for flow cytometry analysis. After 6 months kidneys were taken for immunohistochemistry. During 6 months, blood serum collection was effectuated once a month, from month 2 after the injection of TMPD up to one week before the animal sacrifice. Serum was used to evaluate the concentration of circulating autoantibodies. Anti-RNA ELISA. Evaluation of autoantibodies against RNA was performed by coating ELISA plates with 50 μl/well of 5 μg/ml mouse RNA or DNA in PBS and incubated overnight at 4° C. Next day, the plates were incubated with 1:50 or 1:100 diluted serum samples in working solution (PBS+0.05% Tween20+1% BSA), and the assay was developed with HRP-labeled goat anti-mouse IgG (Southern Biotech).

### Cytokine Shock Model

Wild type female mice C57BL/6 of 8 weeks of age were injected i.p. with 2 mg/kg of GBZ, clonidine or PBS/DMSO. One hour later, they were injected i.p. with 20 mg/mouse of d-GalN and ODN 1826 50 μg/20 g (or PBS). Mice survival was checked every 12 hours for up to 48 hours. For some experiments, blood sera were collected 1 and 3 h after the injection of D-galN. The serum was used to measure circulating cytokines (TNFα, IL-6, IFNβ IL-10, IL-12). For some experiments, the liver was collected for immunohistochemistry after two days. For i.p. injections, 1 ml syringes and 25 gauge needles were used. Blood was collected from the cheek with a 18 gauge needle; for the TMPD model, no more than 200 μL were collected each time, respecting the ethic statement.

### Tissue Histology

Mouse kidney and liver were fixed in 10% buffered formalin for 24 h, then the tissues were dehydrated and embedded in paraffin. Tissue sections of 3.5 μm were cut using the microtom Leica RM2245. Hematoxylin-eosin staining was effectuated automatically with Leica autostainer XL. Finally, the slides were mounted with entellan and kept at room temperature. Biopsies were analyzed by an anatomopathologist and the clinical score was assigned in a blinded way. Pictures were taken with Nikon Eclipse.

### Anatomo-Pathology Scoring System for Liver Damage

a) Lobular inflammation
0—no inflammation, 1—mild lobular inflammation (<10% of liver parenchyma), 2—moderate lobular inflammation (10-50% of liver parenchyma), 3—severe lobular inflammation (>50% of liver parenchyma)
b) Portal inflammation
0—no portal inflammation, 1—mild portal inflammation (<⅓ of portal tracts), 2—moderate portal inflammation (approximately 50% of portal tracks), 3—severe portal inflammation (>⅔ of portal tracts)
Portal and lobular inflammation score are added=inflammation score
c) Necrosis
0—No necrosis, 1—<10% necrosis of liver parenchyma, 2—10-25% necrosis of liver parenchyma, 3—25% necrosis of liver parenchyma
Inflammation score and necrosis score are added=Total histologic score

### Statistical Analysis

Statistical analysis was performed with GraphPad Prism software. When several conditions were to compare, we performed a one-way ANOVA, followed by Tukey range test to assess the significance among pairs of conditions. When only two conditions were to test, we performed Student's t-test or Welch t-test (according the validity of homoscedasticity hypothesis). Statistical analysis on survival curves were performed using Log-rank test, followed by Benjamini-Yekutieli correction when required.

### Results

### Guanabenz Treatment Partially Phenocopies GADD34 Genetic Inactivation in DCs

We have previously shown that GM-CSF-induced bone-marrow-derived DCs from GADD34^{ΔC/ΔC} mouse display limited capacity to express IFNβ and IL-6 in response to the dsRNA mimic polyriboinosinic:polyribocytidylic acid (poly(I:C)). We investigated whether this observation could be extended to other DC subsets and TLR ligands. WT and GADD34^{ΔC/ΔC} DC isolated directly from spleen or generated in vitro from bone-marrow progenitors, using either Flt3L- or GM-CSF-induced differentiation, were submitted to stimulation with LPS, poly(I:C) or CpG ODN and analyzed for IFN-β and IL-6 production (data not shown). Flt3L-DCs are well suited for these in vitro experiments since they encompass all the main DCs subsets (DC1, DC2 and pDCs) found in vivo and express preferentially either TLR3, TLR4 or TLR9. After poly(I:C) stimulation, Flt3L-DCs showed reduced IFN-β and IL-6 production in absence of functional GADD34 (data not shown), confirming the cytokine deficit observed in GADD34^{ΔC/ΔC} GM-CSF-DCs (data not shown). Interestingly, IFN-β production in response to LPS, which remained unaffected by GADD34 deficiency in GM-DCs, was augmented in GADD34^{ΔC/ΔC} Flt3L-DCs (data not shown), and the converse situation was true for IL-6 secretion (data not shown). When compared to in vitro generated cells, CD11c⁺ DCs directly isolated from mouse spleen were also sensitive to GADD34 inactivation (data not shown), IL-6 production being however less effectively inhibited, suggesting that GADD34 impact varies according to the cell type studied and the stimulation or differentiation conditions used.

We next tested if pharmacological treatment with Guanabenz (GBZ, 50 μM), a small proteostatic compound previously described to inhibit GADD34/PP1c interaction, could replicate the impact of Gadd34 genetic inactivation on DC (data not shown). For IL-6 production, in most cases GBZ had an activity superimposable to GADD34 deficiency. However, we noticed that GBZ had a far stronger capacity to inhibit IFN-β production in response to TLR9 stimulation than Gadd34 genetic inactivation. This effect was particularly acute upon CpG ODN stimulation of GADD34^{ΔC/ΔC} Flt3L-DCs, which were incapable of producing IFN-β, nor IL-6 upon GBZ treatment, which also synergized with the lack of functional GADD34. These additive effects also suggested that GBZ interferes with proteostasis independently of GADD34/PP1c binding, as recently proposed by Crespillo-Casado and collaborators.

### Guanabenz is a Strong Inhibitor of DC Activation by Poly(I:C) and CpG ODN

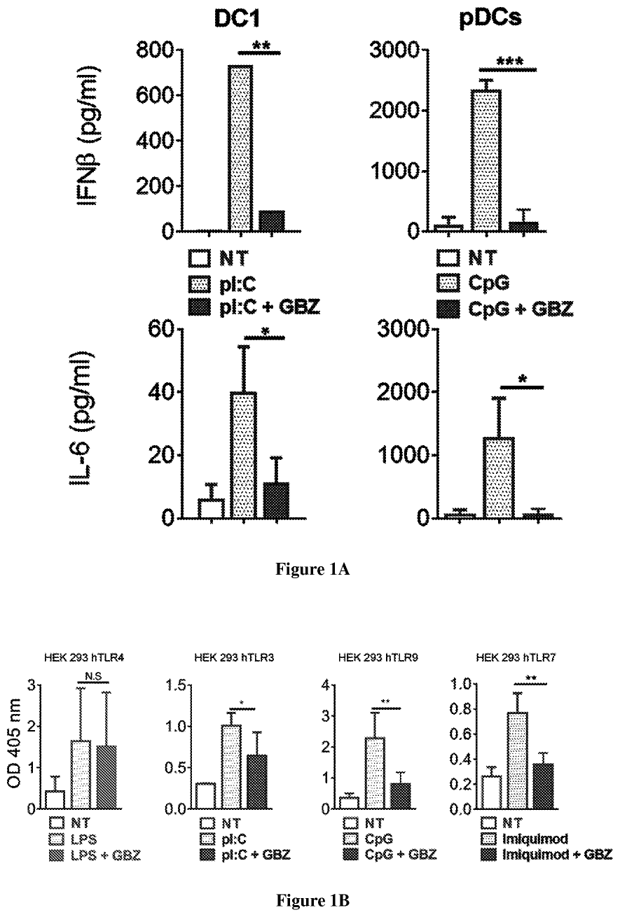

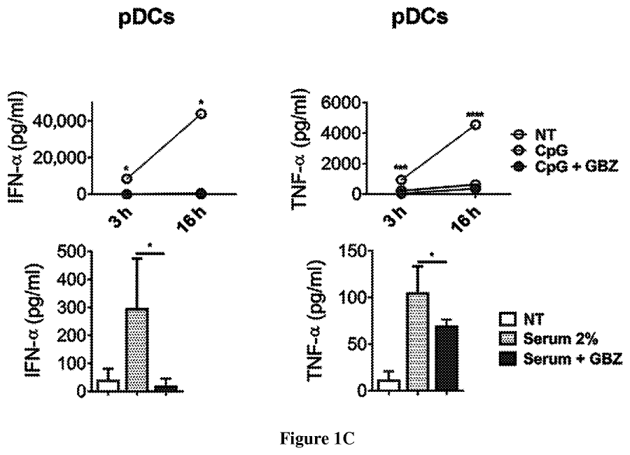

The intensity of TLR3 and TLR9 inhibition caused by GBZ was particularly striking and we decided to investigate further this effect. Highest expression of TLR3 and TLR9 among mouse DC subsets is found respectively in CD8⁺ like conventional DC (cDC1) and in pDC. Mouse CD11c⁺/MHCII⁺ DCs from Flt3L-treated bone marrow cultures were sorted as, cDC1 (CD24⁺/Sirpα⁻), cDC2 (CD11b⁺/Sirpα⁺) and pDC (BST2⁺/Siglec H⁺/B220⁺) (data not shown). Sorted TLR3-expressing cDC1 were then activated with low molecular weight (LMW) poly (I:C), while TLR9-expressing pDCs were stimulated with CpG-A ODN in the presence or absence of GBZ. In both cases, GBZ treatment nearly abrogated cytokines production and particularly IFN-β and IL-6 secretion by pDCs (FIG. 1A), without any impact on cell fitness. GBZ also inhibited completely the transcriptional response to CpG ODN, as judged by the loss of key gene expression in pDC, including ifna4, Tnfa, isg15, and Il12, all monitored by qPCR (data not shown).

Given the efficiency of GBZ on mouse TLR3 and TLR9, we turned to human TLRs, in order to extend the relevance of our observations. GBZ-mediated inhibition was first evaluated on NF-kB signaling by monitoring secreted alkaline phosphatase (SEAP) reporter levels in HEK-293 cells expressing TLR3, TLR4, TLR7, TLR9 (FIG. 1B). As expected, 50 μM of GBZ treatment inhibited TLR3-, TLR7- and TLR9-dependent activation of the SEAP reporter, but not of TLR4. We next treated freshly isolated BDCA4⁺/CD123⁺ human primary blood pDCs activated with either CpG-A ODN or DNA-IC-containing serum from lupus patients (FIG. 1C). GBZ prevented the production of type-I IFN and TNF-α by stimulated human pDCs independently of the ligands used. We could extend these observations to both the human CAL-1 pDCs cell line (data not shown) and primary B cells, from either mouse (data not shown) or human origin (data not shown). The functionality of the TLR9 transduction pathway was next evaluated by using phospho-flow detection of 40S ribosomal protein S6 (rpS6) phosphorylation, a well characterized early signaling event down-stream of TLR activation by LPS or viruses. GBZ-treated CAL-1 and human B cells both displayed a strong reduction in rpS6 phosphorylation normally induced after 15 min to 1 h of CpG exposure (data not shown), suggesting that GBZ inhibits an upstream step of the TLR9 signal transduction pathway.

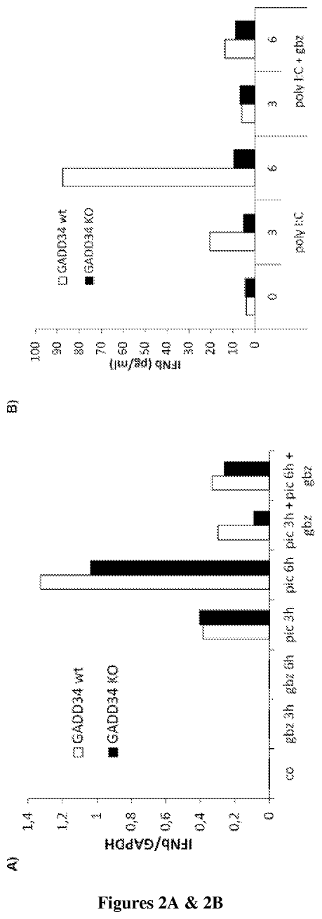

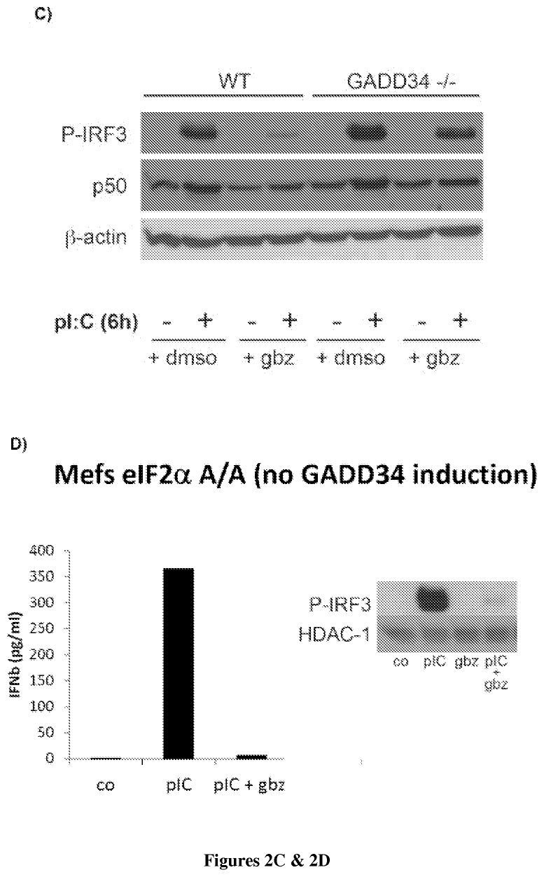

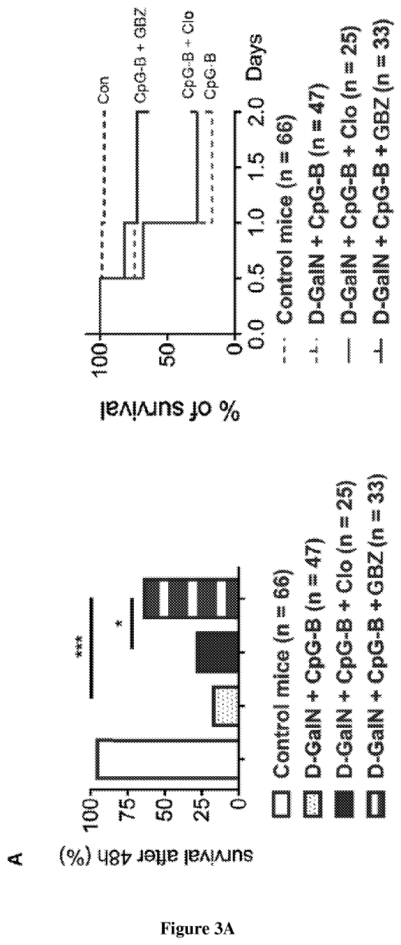

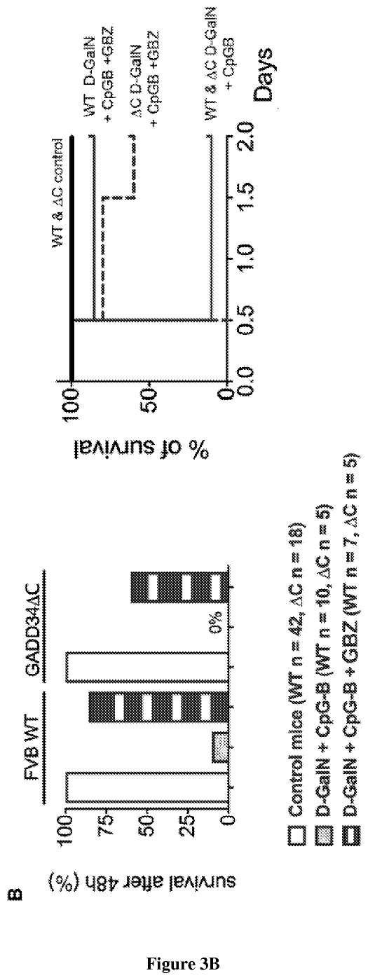

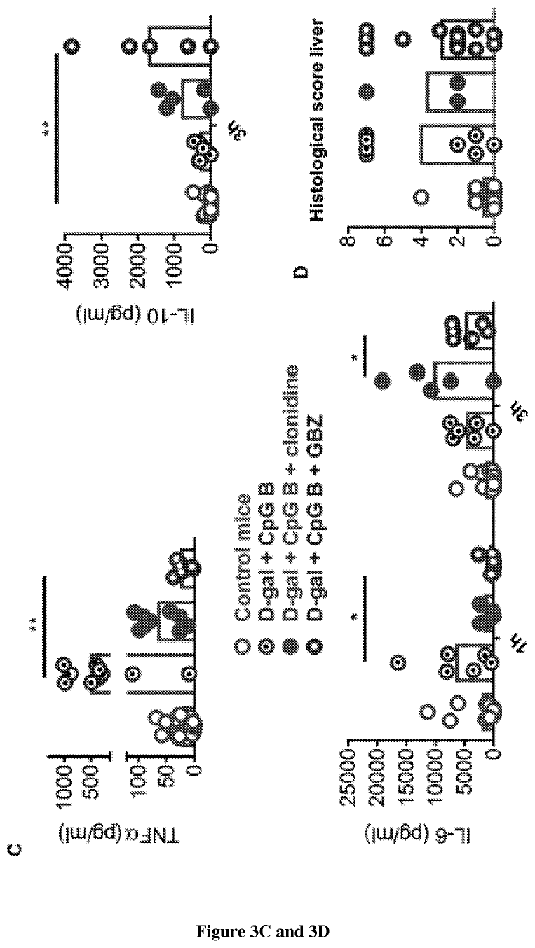

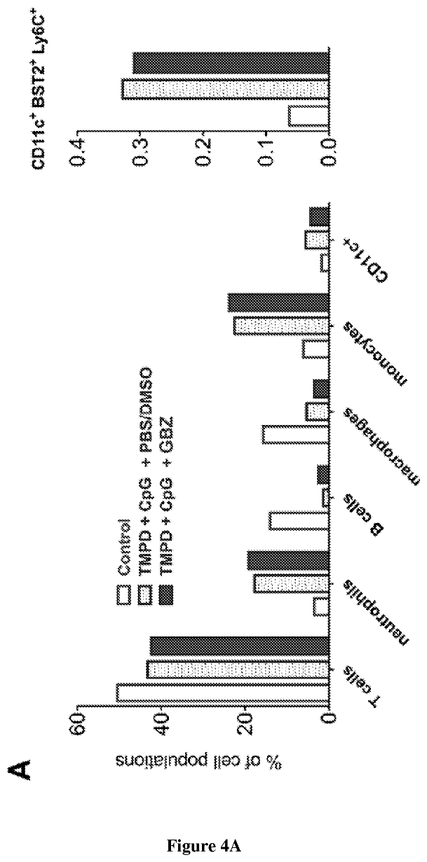

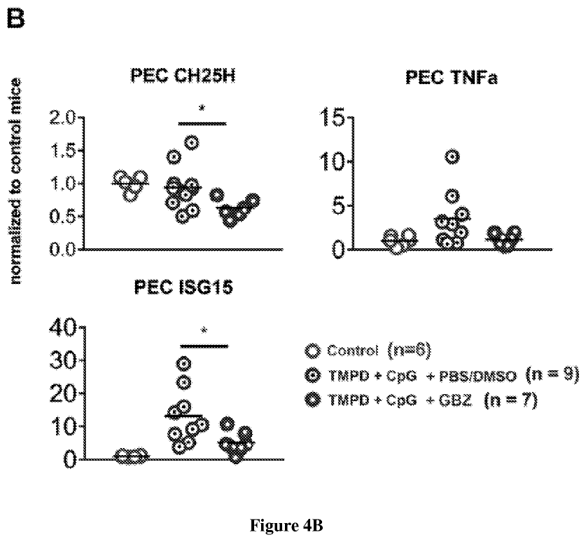

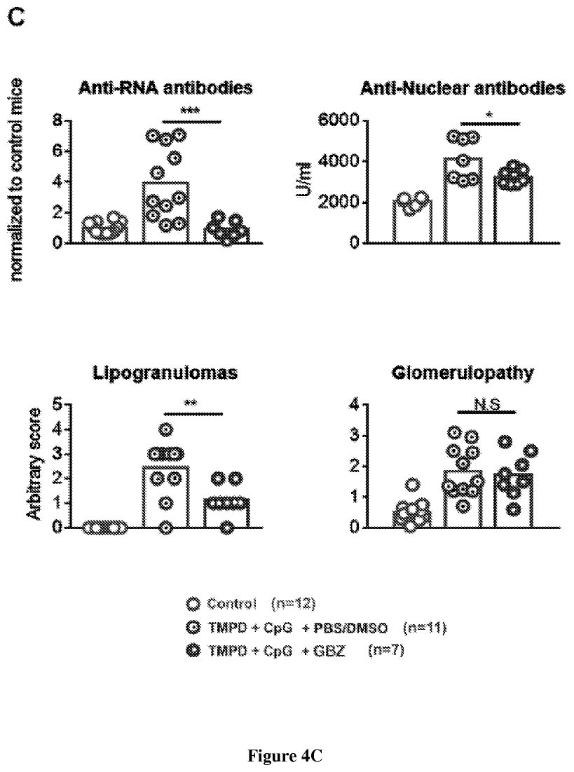

Interestingly GBZ also inhibited the activation of IRF3 and subsequent transcription of type I IFN-β upon lipofection with poly(I:C) of mouse embryonic fibroblasts, indicating that GBZ also inhibits cytosolic RIG I-like receptor activation by nucleic acids (FIG. 2).

Guanabenz Protects Mouse from CpG Induced Cytokine Shock Independently of GADD34

In addition of its hypotensive indications for human pathologies, the therapeutic potential of Guanabenz for misfolded protein stress-associated diseases as been tested in mouse. The primary focus of these experiments was to evaluate GBZ impact on neurodegenerative diseases, characterized by both neuron degeneration and inflammation. Despite contrasting results, these experiments in mouse have yielded important pharmacokinetics data on GBZ, including a plasma half-life of 1.8 hours and a spinal cord accumulation of approximately 7 μM, several hours following single intraperitoneal (i.p.) bolus injection of 2 mg/kg. We decided to test GBZ inhibitory activity on TLR9 in vivo, by taking advantage of the D-galactosamine (D-galN) liver sensitization model followed by CpG ODN injection (FIG. 3A). The survival rate of CpG injected animals was inferior to 20% after 48 h. This value was not substantially changed when animals were co-injected with clonidine (N-(2,6-dichlorophényl)-4,5-dihydro-1H-imidazol-2-amine), a different a2-adrenergic receptor agonist without GADD34 inhibitory activity. Clonidine was therefore used as control compound throughout our study to evaluate the impact of a2-adrenergic inhibitory receptor signaling and hypotension on the general toxicity induced by D-galN and CpG injections. When GBZ (2 mg/kg) was co-injected with CpG, a dramatic rescue of animal's viability was observed, with values reaching up to 70% of survival after 48 h. Importantly, when GADD34^{ΔC/ΔC} mice were examined, they displayed the same sensitivity than WT animals to D-galN and CpG injections, and were also protected from death by GBZ delivery, despite GADD34 inactivation (FIG. 3B). GBZ exerts therefore a strong inhibitory activity over TLR9 in vivo, this independently of GADD34, although with a decreased efficiency (˜28%), suggesting nevertheless the existence of some cross-talks between GBZ-induced proteostasis and GADD34 inactivation to favor survival in this pathological model.

In WT animals, circulating TNF-α and IL-6 plasma concentrations, monitored 1 hour after CpG injection, were strongly attenuated by GBZ-treatment (FIG. 3C). Interestingly, although circulating type-I IFN remained undetectable, production of the anti-inflammatory cytokine IL-10 was strongly increased, suggesting that GBZ protective effect could also be exerted by reinforcing secondary anti-inflammatory responses in vivo (FIG. 3C). Although much less efficient than GBZ, clonidine had some inhibitory effect on TNF-α and IL-6 production after 1 h, but this incapacitation was not observed after 3 h (FIG. 3C), suggesting that hypotension could contribute to reduce inflammatory cytokines diffusion for a short period after injection. In the D-galN model, LPS-induced lethality has been shown to be triggered by a caspase-dependent fulminant apoptotic hepatitis induced by TNF-α over-production not directly from the systemic inflammatory response. However, in this model of CpG-induced lethality, liver examination and histological scores indicated that GBZ had little direct protective effect on liver cells survival (FIG. 3D), and that its activity was most likely exerted through inhibition of pro-inflammatory cytokines release by immunocytes and not by hepatocytes.

Guanabenz Protects Mouse from TMPD-Induced Auto-Immunity.

Tetramethylpentadecane (TMPD or pristane) intraperitoneal injection is used to induce a systemic lupus erythematosus (SLE)-like disease in mouse. In this pathological model, glomerulonephritis and autoantibody production strictly depend on signaling through the type-I IFN receptor (IFNAR) and the formation of “lipogranulomas”, which represent a chronic inflammatory response to TMPD and are the sites of autoantibodies production. Monocytes recruited to the inflamed peritoneal cavity, and their activation by endogenous TLR7 and potentially TLR9 ligands, are the major sources of type-I IFN in this model, which after a period of 3 to 4 month, results in different auto-immune manifestations, specific to the mouse genetic background studied. The TMPD-inducible SLE model appears therefore to be particularly adapted to test GBZ inhibitory activity on endosomal TLRs in vivo, since it is inducible, type-I IFN dependent and mimics some of human SLE features. We modified the original protocol of TMPD-induced SLE by adding an i.p. injection of CpG ODN, in order to boost TLR9-dependent activation and reinforce type-I IFN production that should be counteracted by GBZ treatment. CpG injection alone had a comparable effect to TMPD on the recruitment of different inflammatory cell populations, in particular of Ly6G⁺ neutrophils and Ly6C⁺ monocytes, which were enriched in day 14 peritoneal exudate cells (PEC) (data not shown), suggesting that the two treatments could synergize, as demonstrated by the increased type-I IFN signature observed immediately after CpG injection (data not shown).

PECs from TMPD/CpG injected C57BL/6 mice, treated with GBZ or not, were analyzed by flow cytometry (FIG. 4A). As expected, TMPD/CpG treatment decreased peritoneal B cells numbers (CD11b^{dim/-}/CD19⁺), while concomitantly increasing neutrophils (Ly6G⁺/CD11b⁺) and inflammatory monocytes (Ly6C⁺) recruitment. GBZ injections did not prevent immune cells migration into or from the peritoneal cavity, indicating that the drug does not impact this key step in TMPD-induced pathology (data not shown and FIG. 4A). We next attempted to measure type-I IFN expression and associated-transcriptional signatures in peritoneal cells of 2 different mouse cohorts (FIG. 4B). Although, we failed to detect directly any consistent induction of IFN-α or IFN-α mRNA, we observed a clear transcriptional up-regulation of TNF-α and of type-I IFN-inducible ISG 15 and CH25H genes in PECs isolated from TMPD+CpG injected mice compared to control littermates. These transcriptional responses, indicative of both an on-going inflammation and type-I IFN production, were however strongly inhibited by recurrent GBZ injections (FIG. 4B). Given the strong bias of TMPD-treated C57BL/6 mice to generate, in the long term, anti-ribonucleoprotein (RNP) or RNA autoantibodies rather than anti-DNA autoantibodies, circulating anti-nuclear and anti-RNA immunoglobulins (Ig) plasma concentration was measured 22 weeks after TMPD injection. Both antibodies titers were substantially lower in GBZ-treated mice than in animals injected with TMPD and CpG only (FIG. 4C). In agreement with these data, considerably smaller numbers of plasma cells-containing lipogranulomas were found in the peritoneal cavity of GBZ-treated mice, explaining the lower circulating autoantibodies concentration and confirming the inhibitory activity of GBZ on endocytic TLRs activity (FIG. 4C). As expected in the C57BL/6 background, and despite additional CpG ODN treatment, we were unable to induce overall sufficient glomerulonephritis to observe significant differences in the levels of Ig-complexes deposits in the kidneys of control and GBZ-treated animals. Taken altogether, these data support the efficacy of Guanabenz, as an inhibitory compound for nucleic acid activation of TLRs in vivo.

### DISCUSSION

Upon induction, GADD34 recruits the catalytic subunit of protein phosphatase 1 (PP1c) to dephosphorylate eIF2α, allowing protein synthesis to resume in a negative feed-back loop that terminates UPR signaling. We demonstrated that GADD34 also regulates pro-inflammatory cytokines and type-I IFN expression, both at the transcriptional and translational level. GADD34 is therefore part of what is described as the “anti-microbial stress response” (MSR), which uses stress-signaling cascades to potentiate innate immune responses. GADD34/PP1c complex has been proposed to act directly on the MAPK and NF-κB signaling by dephosphorylating TGF-beta-activated kinase 1 (TAK1) at serine 412 (Ser412), with the consequent inhibition of TNF-α and IL-6 production upon TLR activation. These data were not subsequently confirmed, but instead, deactivation of the kinase IKK by GADD34/PP1 was proposed as an alternative to control LPS activation in macrophages. GADD34 expression in macrophages also enhances autophagy and suppresses apoptosis through regulation of mTOR signaling pathway upon combined LPS stimulation and amino acid deprivation. It is thus likely that accordingly to the immune-stimulation and the cell types studied, major variations in the responses of Gadd34-deficient cell and animals can be observed.

Guanabenz (GBZ), an a2-adrenergic receptor agonist used in the treatment of hypertension, has been proposed to selectively disrupts the stress-induced de-phosphorylation of eIF2α by GADD34/PP1, without affecting the related CReP-phosphatase complex. We have shown here that the proteostasis-interfering drug GBZ inhibits very efficiently endosomal TLR signaling in vitro and mimics partially GADD34-genetic inactivation by controlling cytokines expression after endocytic TLR stimulation. However, in agreement with the recent observation by Crespillo-Casado and collaborators, we could demonstrate that GBZ exerts its inhibitory activity on TLR9 independently of GADD34 function. The capacity of GBZ to block endocytic TLRs in both pDCs and B cells, two major cellular actors of autoimmunity, suggests that this drug could also be used to treat different acute inflammatory diseases. The hypotensive activity of GBZ is clearly not involved in the drug efficacy on TLR signaling, and, on one hand, could constitute globally a handicap in the clinic for many potential novel indications. On the other hand, fifty percent of SLE patients experience hypertension generally caused by obesity, kidney disease, and importantly by long-term steroid use. Thus, GBZ effect on hypertension, taken together with its capacity to inhibit TLR9 and may be TLR7, could, in the long run, be highly beneficial for patients with SLE or other specific IFN-dependent diseases displaying similar pathologies with recurrent flairs.

Novel GBZ-like drugs, designed to lack a2-adrenergic receptor agonistic activity (WO2016/001390), are now becoming available and currently being tested to treat neurodegenerative conditions. The combined capacity of these drugs to interfere both with proteostasis and immunity might become a double edge advantage for the treatment of neurodegenerative diseases, which often combine increased neuronal death due to misfolded proteins accumulation, accompanied by acute inflammation that further triggers tissue damages and contributes to disease progression. Such dual effect also explains the protective role of GBZ treatment in DSS-induced colitis and ulcerative colitis induction in mouse that involve both inflammatory cytokines release and epithelial cells stress response.

Guanabenz, thus represents a novel pharmacological option for the treatment of type-I interferon-dependent diseases. By reducing protein synthesis restoration during stress and changing the capacity of TLRs to signal in immune cells, GBZ-like compounds could have the capacity to treat both proteotoxicity and associated inflammatory responses, which are key features of autoimmunity and neurodegeneration. Our findings that GBZ exerts its activity independently of GADD34, does not however diminish the attractiveness of GADD34/PP1c, as a drug-able target to alter cytokines production and proteostasis, potentially offering in the future, yet another alternative solution to treat auto-inflammatory diseases, provided that small and specific pharmacological inhibitory compounds can be isolated.

### REFERENCES

Throughout this application, various references describe the state of the art to which this invention pertains. The disclosures of these references are hereby incorporated by reference into the present disclosure.
S. Akira, S. Uematsu, O. Takeuchi, Pathogen recognition and innate immunity. Cell 124, 783-801 (2006).
T. Kawai, S. Akira, The role of pattern-recognition receptors in innate immunity: update on Toll-like receptors. Nat Immunol 11, 373-384 (2010).
S. E. Ewald, A. Engel, J. Lee, M. Wang, M. Bogyo, G. M. Barton, Nucleic acid recognition by Toll-like receptors is coupled to stepwise processing by cathepsins and asparagine endopeptidase. J Exp Med 208, 643-651 (2011).
F. E. Sepulveda, S. Maschalidi, R. Colisson, L. Heslop, C. Ghirelli, E. Sakka, A. M. Lennon-Dumenil, S. Amigorena, L. Cabanie, B. Manoury, Critical role for asparagine endopeptidase in endocytic Toll-like receptor signaling in dendritic cells. Immunity 31, 737-748(2009).
S. Bauer, C. J. Kirschning, H. Hacker, V. Redecke, S. Hausmann, S. Akira, H. Wagner, G. B. Lipford, Human TLR9 confers responsiveness to bacterial DNA via species-specific CpG motifrecognition. Proc Natl Acad Sci USA 98, 9237-9242 (2001).
H. Hemmi, O. Takeuchi, T. Kawai, T. Kaisho, S. Sato, H. Sanjo, M. Matsumoto, K. Hoshino, H. Wagner, K. Takeda, S. Akira, A Toll-like receptor recognizes bacterial DNA. Nature 408, 740-745 (2000).
M. Gilliet, W. Cao, Y. J. Liu, Plasmacytoid dendritic cells: sensing nucleic acids in viral infection and autoimmune diseases. Nat Rev Immunol 8, 594-606 (2008).
K. Honda, Y. Ohba, H. Yanai, H. Negishi, T. Mizutani, A. Takaoka, C. Taya, T. Taniguchi, Spatiotemporal regulation of MyD88-IRF-7 signalling for robust type-I interferon induction. Nature 434, 1035-1040 (2005).
R. Lande, D. Ganguly, V. Facchinetti, L. Frasca, C. Conrad, J. Gregorio, S. Meller, G. Chamilos, R. Sebasigari, V. Riccieri, R. Bassett, H. Amuro, S. Fukuhara, T. Ito, Y. J. Liu, M. Gilliet, Neutrophils activate plasmacytoid dendritic cells by releasing self-DNA-peptide complexes in systemic lupus erythematosus. Sci Transl Med 3, 73ra19 (2011).
J. E. Gottenberg, N. Cagnard, C. Lucchesi, F. Letourneur, S. Mistou, T. Lazure, S. Jacques, N. Ba, M. Ittah, C. Lepajolec, M. Labetoulle, M. Ardizzone, J. Sibilia, C. Fournier, G. Chiocchia, X. Mariette, Activation of IFN pathways and plasmacytoid dendritic cell recruitment in target organs of primary Sjogren's syndrome. Proc Natl Acad Sci USA 103, 2770-2775 (2006).
D. Ron, P. Walter, Signal integration in the endoplasmic reticulum unfolded protein response. Nat Rev Mol Cell Biol 8, 519-529 (2007); published online EpubJul (10.1038/nrm2199).
N. Claudio, A. Dalet, E. Gatti, P. Pierre, Mapping the crossroads of immune activation and cellular stress response pathways. EMBO J 32, 1214-1224 (2013).
G. Clavarino, N. Claudio, T. Couderc, A. Dalet, D. Judith, V. Camosseto, E. K. Schmidt, T. Wenger, M. Lecuit, E. Gatti, P. Pierre, Induction of GADD34 is necessary for dsRNA-dependent interferon-beta production and participates in the control of Chikungunya virus infection. PLoS Pathog 8, e1002708 (2012).
G. Clavarino, N. Claudio, A. Dalet, S. Terawaki, T. Couderc, L. Chasson, M. Ceppi, E. K. Schmidt, T. Wenger, M. Lecuit, E. Gatti, P. Pierre, Protein phosphatase 1 subunit Ppp1r15a/GADD34 regulates cytokine production in polyinosinic:polycytidylic acid-stimulated dendritic cells. Proc Natl Acad Sci USA 109, 3006-3011 (2012).
P. Walter, D. Ron, The unfolded protein response: from stress pathway to homeostatic regulation. Science 334, 1081-1086 (2011).
I. Novoa, H. Zeng, H. P. Harding, D. Ron, Feedback inhibition of the unfolded protein response by GADD34-mediated dephosphorylation of eIF2alpha. J Cell Biol 153, 1011-1022 (2001).
I. Novoa, Y. Zhang, H. Zeng, R. Jungreis, H. P. Harding, D. Ron, Stress-induced gene expression requires programmed recovery from translational repression. EMBO J 22, 1180-1187 (2003).
S. Ito, Y. Tanaka, R. Oshino, S. Okado, M. Hori, K. I. Isobe, GADD34 suppresses lipopolysaccharide-induced sepsis and tissue injury through the regulation of macrophage activation. Cell Death Dis 7, e2219 (2016).
P. Tsaytler, H. P. Harding, D. Ron, A. Bertolotti, Selective inhibition of a regulatory subunit of protein phosphatase 1 restores proteostasis. Science 332, 91-94 (2011).
I. Das, A. Krzyzosiak, K. Schneider, L. Wrabetz, M. D'Antonio, N. Barry, A. Sigurdardottir, A. Bertolotti, Preventing proteostasis diseases by selective inhibition of a phosphatase regulatory subunit. Science 348, 239-242 (2015).
B. Holmes, R. N. Brogden, R. C. Heel, T. M. Speight, G. S. Avery, Guanabenz. A review of its pharmacodynamic properties and therapeutic efficacy in hypertension. Drugs 26, 212-229 (1983).
S. W. Way, J. R. Podojil, B. L. Clayton, A. Zaremba, T. L. Collins, R. B. Kunjamma, A. P. Robinson, P. Brugarolas, R. H. Miller, S. D. Miller, B. Popko, Pharmaceutical integrated stress response enhancement protects oligodendrocytes and provides a potential multiple sclerosis therapeutic. Nat Commun 6, 6532 (2015).
A. Crespillo-Casado, J. E. Chambers, P. M. Fischer, S. J. Marciniak, D. Ron, PPP1R15A-mediated dephosphorylation of eIF2alpha is unaffected by Sephin1 or Guanabenz. Elife 6, (2017).
H. P. Harding, Y. Zhang, D. Scheuner, J. J. Chen, R. J. Kaufman, D. Ron, Ppp1r15 gene knockout reveals an essential role for translation initiation factor 2 alpha (eIF2alpha) dephosphorylation in mammalian development. Proc Natl Acad Sci USA 106, 1832-1837 (2009).
W. H. Reeves, P. Y. Lee, J. S. Weinstein, M. Satoh, L. Lu, Induction of autoimmunity by pristane and other naturally occurring hydrocarbons. Trends Immunol 30, 455-464 (2009).
S. H. Naik, A. I. Proietto, N. S. Wilson, A. Dakic, P. Schnorrer, M. Fuchsberger, M. H. Lahoud, M. O'Keeffe, Q. X. Shao, W. F. Chen, J. A. Villadangos, K. Shortman, L. Wu, Cutting edge: generation of splenic CD8+ and CD8− dendritic cell equivalents in Fms-like tyrosine kinase 3 ligand bone marrow cultures. J Immunol 174, 6592-6597 (2005).
M. Guilliams, C. A. Dutertre, C. L. Scott, N. McGovern, D. Sichien, S. Chakarov, S. Van Gassen, J. Chen, M. Poidinger, S. De Prijck, S. J. Tavernier, I. Low, S. E. Irac, C. N. Mattar, H. R. Sumatoh, G. H. Low, T. J. Chung, D. K. Chan, K. K. Tan, T. L. Hon, E. Fossum, B. Bogen, M. Choolani, J. K. Chan, A. Larbi, H. Luche, S. Henri, Y. Saeys, E. W. Newell, B. N. Lambrecht, B. Malissen, F. Ginhoux, Unsupervised High-Dimensional Analysis Aligns Dendritic Cells across Tissues and Species. Immunity 45, 669-684 (2016).
T. Maeda, K. Murata, T. Fukushima, K. Sugahara, K. Tsuruda, M. Anami, Y. Onimaru, K. Tsukasaki, M. Tomonaga, R. Moriuchi, H. Hasegawa, Y. Yamada, S. Kamihira, A novel plasmacytoid dendritic cell line, CAL-1, established from a patient with blastic natural killer cell lymphoma. Int J Hematol81, 148-154 (2005).
C. Guiducci, C. Ghirelli, M. A. Marloie-Provost, T. Matray, R. L. Coffman, Y. J. Liu, F. J. Barrat, V. Soumelis, PI3K is critical for the nuclear translocation of IRF-7 and type I IFN production by human plasmacytoid predendritic cells in response to TLR activation. J Exp Med 205, 315-322(2008).
H. Lelouard, E. K. Schmidt, V. Camosseto, G. Clavarino, M. Ceppi, H. T. Hsu, P. Pierre, Regulation of translation is required for dendritic cell function and survival during activation. J Cell Biol 179, 1427-1439 (2007).
F. G. Vieira, Q. Ping, A. J. Moreno, J. D. Kidd, K. Thompson, B. Jiang, J. M. Lincecum, M. Z. Wang, G. S. De Zutter, V. R. Tassinari, B. Levine, T. Hatzipetros, A. Gill, S. Perrin, Guanabenz Treatment Accelerates Disease in a Mutant SOD1 Mouse Model of ALS. PLoS One 10, e0135570 (2015).
H. Q. Jiang, M. Ren, H. Z. Jiang, J. Wang, J. Zhang, X. Yin, S. Y. Wang, Y. Qi, X. D. Wang, H. L. Feng, Guanabenz delays the onset of disease symptoms, extends lifespan, improves motor performance and attenuates motor neuron loss in the SOD1 G93A mouse model of amyotrophic lateral sclerosis. Neuroscience 277, 132-138 (2014).
R. R. Ruffolo, Jr., A. J. Nichols, J. M. Stadel, J. P. Hieble, Structure and function of alpha-adrenoceptors. Pharmacol Rev 43, 475-505 (1991).
A. Mignon, N. Rouquet, M. Fabre, S. Martin, J. C. Pages, J. F. Dhainaut, A. Kahn, P. Briand, V. Joulin, LPS challenge in D-galactosamine-sensitized mice accounts for caspase-dependent fulminant hepatitis, not for septic shock. Am J Respir Crit Care Med 159, 1308-1315 (1999).
D. C. Nacionales, J. S. Weinstein, X. J. Yan, E. Albesiano, P. Y. Lee, K. M. Kelly-Scumpia, R. Lyons, M. Satoh, N. Chiorazzi, W. H. Reeves, B cell proliferation, somatic hypermutation, class switch recombination, and autoantibody production in ectopic lymphoid tissue in murine lupus. J Immunol 182, 4226-4236 (2009).
P. Y. Lee, Y. Kumagai, Y. Li, O. Takeuchi, H. Yoshida, J. Weinstein, E. S. Kellner, D. Nacionales, T. Barker, K. Kelly-Scumpia, N. van Rooijen, H. Kumar, T. Kawai, M. Satoh, S. Akira, W. H. Reeves, TLR7-dependent and FcgammaR-independent production of type I interferon in experimental mouse lupus. J Exp Med 205, 2995-3006 (2008).
L. Bossaller, A. Christ, K. Pelka, K. Nundel, P. I. Chiang, C. Pang, N. Mishra, P. Busto, R. G. Bonegio, R. E. Schmidt, E. Latz, A. Marshak-Rothstein, TLR9 Deficiency Leads to Accelerated Renal Disease and Myeloid Lineage Abnormalities in Pristane-Induced Murine Lupus. J Immunol 197, 1044-1053 (2016).
H. Zhuang, C. Szeto, S. Han, L. Yang, W. H. Reeves, Animal Models of Interferon Signature Positive Lupus. Front Immunol 6, 291 (2015)10.3389/fimmu.2015.00291).
A. Savina, P. Vargas, P. Guermonprez, A. M. Lennon, S. Amigorena, Measuring pH, ROS production, maturation, and degradation in dendritic cell phagosomes using cytofluorometry-based assays. Methods Mol Biol 595, 383-402 (2010).
P. Pierre, I. Shachar, D. Matza, E. Gatti, R. A. Flavell, I. Mellman, Invariant chain controls H2-M proteolysis in mouse splenocytes and dendritic cells. J Exp Med 191, 1057-1062 (2000).
E. K. Schmidt, G. Clavarino, M. Ceppi, P. Pierre, SUnSET, a nonradioactive method to monitor protein synthesis. Nat Methods 6, 275-277 (2009).
A. R. Tall, L. Yvan-Charvet, Cholesterol, inflammation and innate immunity. Nat Rev Immunol 15, 104-116 (2015).
E. S. Gold, A. H. Diercks, I. Podolsky, R. L. Podyminogin, P. S. Askovich, P. M. Treuting, A. Aderem, 25-Hydroxycholesterol acts as an amplifier of inflammatory signaling. Proc Nat Acad Sci USA 111, 10666-10671 (2014).
S. Hannedouche, J. Zhang, T. Yi, W. Shen, D. Nguyen, J. P. Pereira, D. Guerini, B. U. Baumgarten, S. Roggo, B. Wen, R. Knochenmuss, S. Noel, F. Gessier, L. M. Kelly, M. Vanek, S. Laurent, I. Preuss, C. Miault, I. Christen, R. Karuna, W. Li, D. I. Koo, T. Suply, C. Schmedt, E. C. Peters, R. Falchetto, A. Katopodis, C. Spanka, M. O. Roy, M. Detheux, Y. A. Chen, P. G. Schultz, C. Y. Cho, K. Seuwen, J. G. Cyster, A. W. Sailer, Oxysterols direct immune cell migration via EBI2. Nature 475, 524-527 (2011).
J. G. Cyster, E. V. Dang, A. Reboldi, T. Yi, 25-Hydroxycholesterols in innate and adaptive immunity. Nat Rev Immunol 14, 731-743 (2014).
M. Blanc, W. Y. Hsieh, K. A. Robertson, K. A. Kropp, T. Forster, G. Shui, P. Lacaze, S. Watterson, S. J. Griffiths, N. J. Spann, A. Meljon, S. Talbot, K. Krishnan, D. F. Covey, M. R. Wenk, M. Craigon, Z. Ruzsics, J. Haas, A. Angulo, W. J. Griffiths, C. K. Glass, Y. Wang, P. Ghazal, The transcription factor STAT-1 couples macrophage synthesis of 25-hydroxycholesterol to the interferon antiviral response. Immunity 38, 106-118 (2013).
M. Gu, C. Ouyang, W. Lin, T. Zhang, X. Cao, Z. Xia, X. Wang, Phosphatase holoenzyme PP1/GADD34 negatively regulates TLR response by inhibiting TAK1 serine 412 phosphorylation. J Immunol 192, 2846-2856 (2014).
H. Y. Li, H. Liu, C. H. Wang, J. Y. Zhang, J. H. Man, Y. F. Gao, P. J. Zhang, W. H. Li, J. Zhao, X. Pan, T. Zhou, W. L. Gong, A. L. Li, X. M. Zhang, Deactivation of the kinase IKK by CUEDC2 through recruitment of the phosphatase PP1. Nat Immunol 9, 533-541 (2008).
S. Ito, Y. Tanaka, R. Oshino, K. Aiba, S. Thanasegaran, N. Nishio, K. Isobe, GADD34 inhibits activation-induced apoptosis of macrophages through enhancement of autophagy. Sci Rep 5, 8327 (2015).
R. M. Ransohoff, How neuroinflammation contributes to neurodegeneration. Science 353, 777-783 (2016).
X. Treton, E. Pedruzzi, C. Guichard, Y. Ladeiro, S. Sedghi, M. Vallee, N. Fernandez, E. Bruyere, P. L. Woerther, R. Ducroc, N. Montcuquet, J. N. Freund, I. Van Seuningen, F. Barreau, A. Marah, J. P. Hugot, D. Cazals-Hatem, Y. Bouhnik, F. Daniel, E. Ogier-Denis, Combined NADPH oxidase 1 and interleukin 10 deficiency induces chronic endoplasmic reticulum stress and causes ulcerative colitis-like disease in mice. PLoS One 9, e101669 (2014).
Y. Tanaka, S. Ito, R. Oshino, N. Chen, N. Nishio, K. Isobe, Effects of growth arrest and DNA damage-inducible protein 34 (GADD34) on inflammation-induced colon cancer in mice. Br J Cancer 113, 669-679 (2015).
M. Gilchrist, V. Thorsson, B. Li, A. G. Rust, M. Korb, J. C. Roach, K. Kennedy, T. Hai, H. Bolouri, A. Aderem, Systems biology approaches identify ATF3 as a negative regulator of Toll-like receptor 4. Nature 441, 173-178 (2006).
E. J. Zmuda, L. Qi, M. X. Zhu, R. G. Mirmira, M. R. Montminy, T. Hai, The roles of ATF3, an adaptive-response gene, in high-fat-diet-induced diabetes and pancreatic beta-cell dysfunction. Mol Endocrinol 24, 1423-1433 (2010).
E. S. Gold, S. A. Ramsey, M. J. Sartain, J. Selinummi, I. Podolsky, D. J. Rodriguez, R. L. Moritz, A. Aderem, ATF3 protects against atherosclerosis by suppressing 25-hydroxycholesterol-induced lipid body formation. J Exp Med 209, 807-817 (2012).
H. Y. Jiang, S. A. Wek, B. C. McGrath, D. Lu, T. Hai, H. P. Harding, X. Wang, D. Ron, D. R. Cavener, R. C. Wek, Activating transcription factor 3 is integral to the eukaryotic initiation factor 2 kinase stress response. Mol Cell Biol 24, 1365-1377 (2004).
Y. Ma, Y. Shimizu, M. J. Mann, Y. Jin, L. M. Hendershot, Plasma cell differentiation initiates a limited ER stress response by specifically suppressing the PERK-dependent branch of the unfolded protein response. Cell Stress Chaperones 15, 281-293 (2010).
G. Dranoff, E. Jaffee, A. Lazenby, P. Golumbek, H. Levitsky, K. Brose, V. Jackson, H. Hamada, D. Pardoll, R. C. Mulligan, Vaccination with irradiated tumor cells engineered to secrete murine granulocyte-macrophage colony-stimulating factor stimulates potent, specific, and long-lasting anti-tumor immunity. Proc Natl Acad Sci US A 90, 3539-3543 (1993).
O. Demaria, P. P. Pagni, S. Traub, A. de Gassart, N. Branzk, A. J. Murphy, D. M. Valenzuela, G. D. Yancopoulos, R. A. Flavell, L. Alexopoulou, TLR8 deficiency leads to autoimmunity in mice. J Clin Invest 120, 3651-3662 (2010).

1. A method of treating a type I IFN-dependent pathology in a subject in need thereof comprising administering to the subject a therapeutically effective amount of Guanabenz or a guanabenz derivative.

2. The method according to claim 1 wherein the subject is a human.

3. The method according to claim 1 wherein the type I IFN-dependent pathology is selected from the group consisting of systemic lupus erythematosus (SLE), Sjögren syndrome, myositis, systemic sclerosis (SSc), Aicardi Goutières syndrome, Influenza A virus (IAV)-induced severe disease, juvenile idiopathic arthritis, rheumatoid arthritis, HIV infection, transplant rejection, in graft versus host disease (GVHD), type 1 diabetes, inflammatory bowel diseases (ulcerative colitis and Crohn's disease), Graves' disease, microscopic polyangiitis, Wegener's granulomatosis, autoimmune thyroid diseases, glomerulonephritis, giant cell arteritis, and periodontal diseases.

4. The method according to claim 1 wherein the type I IFN-dependent pathology is systemic lupus erythematosus.

5. The method according to claim 1, wherein the guanabenz derivative is a mono-halogenated (hetero)aryl derivative.

6. The method according to claim 1, wherein the guanabenz derivative is a compound of formula (I): 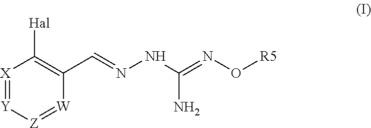
wherein:
Hal=F, Cl, Br, I;
X is either —CR1═ or —N═;
Y is either —CR2═ or —N═;
Z is either —CR3═ or —N═;
W is either —CR4═ or —N═;
R1 is selected from H, Hal, alkyl and O-alkyl;
R2 is selected from H, Hal, alkyl, O-alkyl and C(O)R6;
R3 is selected from H, Hal, alkyl and O-alkyl;
R4 is H, CI, F, I or Br;
R5 is H or alkyl, cycloalkyl, aralkyl, alkenyl, cycloalkenyl, heterocyclyl, aryl, C(O)-alkyl, and C(O)-aryl, each of which is optionally substituted with one or more R7 groups; R6 is selected from OH, O-alkyl, O-aryl, aralkyl, NH₂, NH-alkyl, N(alkyl)₂, NH-aryl, CF₃, alkyl and alkoxy;
each R7 is independently selected from halogen, OH, CN, COO-alkyl, aralkyl, heterocyclyl, S-alkyl, SO-alkyl, SO₂-alkyl, SO₂-aryl, COOH, CO-alkyl, CO-aryl, NH₂, NH-alkyl, N(alkyl)₂, CF₃, alkyl and alkoxy;
and wherein if Hal is CI and R4 is CI, then R5 is not H.

7. The method according to claim 1, wherein the guanabenz derivative is a compound of formula (II): 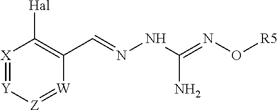
wherein:
Hal=F, Cl, Br, I;
X is either —CR1═ or —N═;
Y is either —CR2═ or —N═;
Z is either —CR3═ or —N═;
W is either —CR4═ or —N═;
R1 is selected from H, Hal, alkyl and O-alkyl;
R2 is selected from H, Hal, alkyl, O-alkyl and C(O)R6;
R3 is selected from H, Hal, alkyl and O-alkyl;
R4 is H, Cl, F, I or Br;
R5 is H or alkyl, cycloalkyl, aralkyl, alkenyl, cycloalkenyl, heterocyclyl, aryl, C(O)-alkyl, and C(O)-aryl, each of which is optionally substituted with one or more R7 groups;
R6 is selected from OH, O-alkyl, O-aryl, aralkyl, NH₂, NH-alkyl, N(alkyl)₂, NH-aryl, CF₃, alkyl and alkoxy;
each R7 is independently selected from halogen, OH, CN, COO-alkyl, aralkyl, heterocyclyl, S-alkyl, SO-alkyl, SO₂-alkyl, SO₂-aryl, COOH, CO-alkyl, CO-aryl, NH₂, NH-alkyl, N(alkyl)₂, CF₃, alkyl and alkoxy.

8. The method according to claim 1, wherein the guanabenz derivative is selected from the group consisting of:

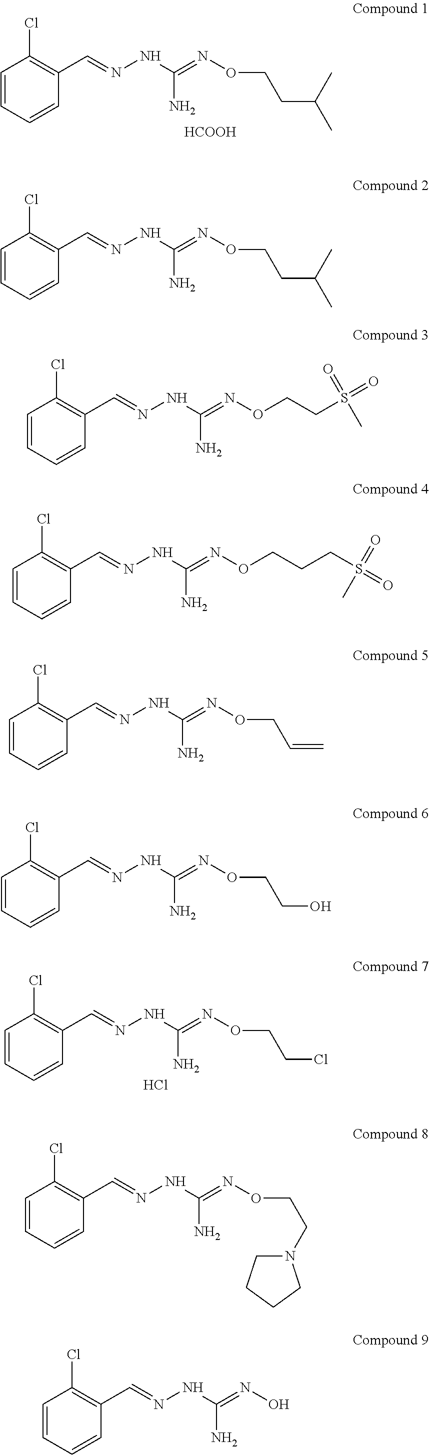

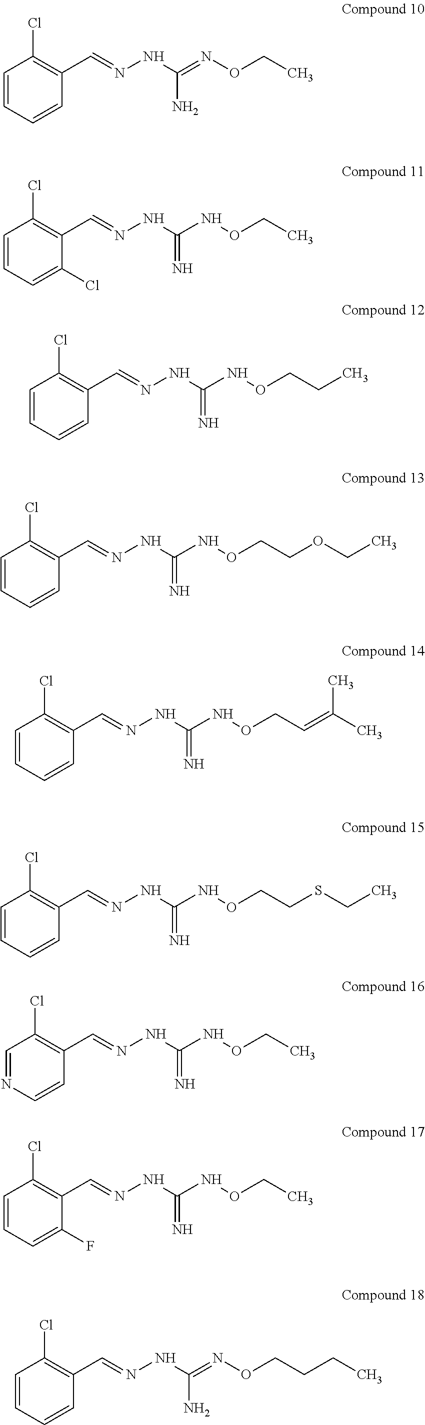

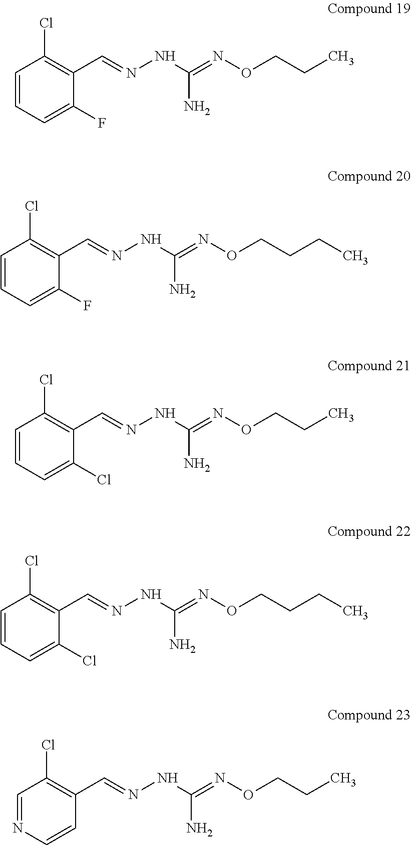 [SMILES: CCCO/N=C(\N)N/N=C/c1c(F)cccc1Cl ; CCCCO/N=C(\N)N/N=C/c1c(F)cccc1Cl ; CCCO/N=C(\N)N/N=C/c1c(Cl)cccc1Cl ; CCCCO/N=C(\N)N/N=C/c1c(Cl)cccc1Cl ; CCCO/N=C(\N)N/N=C/c1ccncc1Cl]

9. The method according to claim 1, wherein the Gaunabenz or derivative thereof is administrated in combination with a standard treatment of type I IFN-dependent pathology.
